(19) United States
(12) Patent Application Publication
(10) Pub. No.: US 2025/0009751 A1
(43) Pub. Date: Jan. 9, 2025

(54) FORMULATIONS OF ANTIVIRAL COMPOUNDS

(71) Applicant: Merck Sharp & Dohme LLC, Rahway, NJ (US)

(72) Inventors: Sutthilug Sotthivirat, Lansdale, PA (US); Joyce Stellabott, Perkasie, PA (US); Walter R. Wasylaschuk, Philadelphia, PA (US)

(73) Assignee: Merck Sharp & Dohme LLC, Rahway, NJ (US)

(21) Appl. No.: 18/893,201

(22) Filed: Sep. 23, 2024

Related U.S. Application Data
(63) Continuation of application No. 17/286,620, filed on Apr. 19, 2021.
(60) Provisional application No. 62/751,262, filed on Oct. 26, 2018.

(51) Int. Cl. A61K 31/5365; A61K 9/20

(52) U.S. Cl. CPC A61K 31/5365; A61K 9/20; A61K 9/2013; A61K 9/2018; A61K 9/2031; A61K 9/2054

(57) ABSTRACT

The present disclosure is directed to pharmaceutical formulations comprising an amorphous inhibitor of hepatitis C virus NS5A. These pharmaceutical formulations may be prepared by roller-compaction or wet-granulation methods. The present disclosure is also directed to oral dosage forms, such as tablets, comprising such pharmaceutical formulations.

### CROSS-REFERENCE TO RELATED APPLICATIONS

This application is a continuation of U.S. patent application Ser. No. 17/286,620, filed Apr. 19, 2021, which is a National Stage Application of International Application No. PCT/US2019/057117, filed Oct. 21, 2019, which claims priority to U.S. Provisional Patent Application No. 62/751,262, filed Oct. 26, 2018. The entire contents of these applications are hereby incorporated herein by reference in their entireties.

### FIELD OF THE INVENTION

The instant invention relates to pharmaceutical formulations that are useful for the treatment of diseases and disorders caused by hepatitis C virus (“HCV”). In particular, the pharmaceutical formulations comprise antiviral compounds that are HCV NS5A inhibitors.

### BACKGROUND OF THE INVENTION

HCV infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin. Several virally encoded enzymes are putative targets for therapeutic intervention, including a metalloprotease (non-structural (NS) 2-3), a serine protease (NS3, amino acid residues 1-180), a helicase (NS3, full length), an NS3 protease cofactor (NS4A), a membrane protein (NS4B), a zinc metalloprotein (NS5A), and an RNA-dependent RNA polymerase (NS5B).

An identified target for therapeutic intervention is the HCV NS5A non-structural protein, which is described, for example, in Seng-Lai Tan & Michael G. Katze, 284 VIROLOGY 1-12 (2001); and in Kyu-Jin Park et al., 278 (33) J. BIO. CHEM. 30711 (2003). A non-structural protein, NS5A is an essential component for viral replication and assembly. Mutations in NS5A at or near known sites of phosphorylation can affect the ability for high-level replication in cell-culture systems, suggesting an important role for NS5A phosphorylation in viral replication efficiency. Inhibitors of the phosphorylation of NS5A can lead to reduced viral RNA replication.

NS5A inhibitor compounds include compounds such as dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, which is shown below as Compound A:

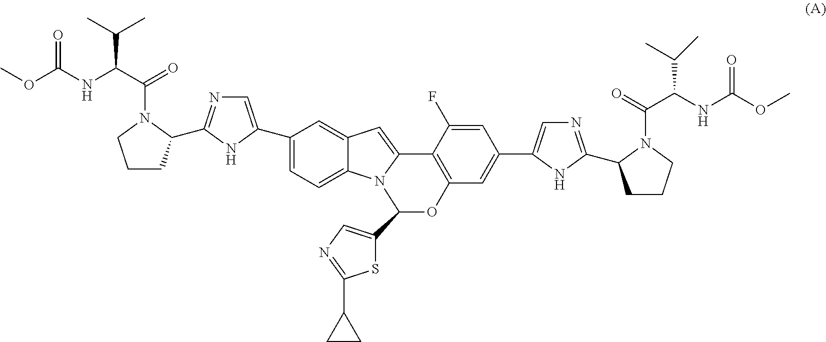

Compound A is described in PCT International Patent Application Publication No. WO2014/110705 and in United States Patent Application Publication No. US2015/0335648.

Compound A, a weak base, has two basic sites, which protonate at low pH giving rise to a sharp pH-dependent solubility profile, particularly between pH 1-4. A normal human stomach has a pH in a range from 1-3, usually closer to 2, although it varies depending on the type and quantity of food ingested. The steep pH-dependent solubility profile has practical implications for dissolution and absorption of Compound A, as for the dissolution and absorption of weak bases in general, in the gastrointestinal tract of patients. Specifically, the amount of drug dissolved from formulations of weakly basic compounds can vary as the gastric pH fluctuates within this normal range, which in turn can lead to more variable and potentially lower absorption. See E. Lahner et al., 29 ALIMENTARY PHARMACOL. THER. 1219-1229 (2009); T. L. Russell et al., 11 (1) PHARM. RES. 136-143 (1994); G. Krishna et al., 53 (3) ANTIMICROB. AGENTS CHEMOTHER. 958-966 (2009).

Patients may exhibit a significantly higher gastric pH, known as achlorhydria, which can arise due to age or concomitant disease, for example, or which can be the result of other drug treatments (e.g., proton pump inhibitors, H2 receptor antagonists). See A. Mitra & F. Kesisoglou, 10 MOL. PHARM. 3970-3979 (2013). Absorption of weakly basic drugs that have low solubility at higher pH (e.g., ketoconazole, itraconazole, atazanavir, cefpodoxime, enoxacin, dipyridamole, nifedipine, and digoxin) has been shown to be impaired due to this condition. See E. Lahner et al., 29 ALIMENTARY PHARMACOL. THER. 1219-1229 (2009).

Because of the importance of gastric pH in driving dissolution, absorption, and ultimately efficacy of Compound A, it is imperative to develop formulations that can minimize or mitigate the effects of increased gastric pH on the bioavailability of Compound A. Such formulations may prove particularly useful in the treatment of HIV patients who are coinfected with HCV. About one-quarter of HIV-infected persons in the United States are also infected with HCV, and these patients tend to have higher gastric pH. See HIV and Viral Hepatitis Fact Sheet, Centers for Disease Control and Prevention (March 2014), available online at http://www.cdc.gov/hepatitis/Populations/PDFs/HIVandHep-FactSheet.pdf. Similarly, these formulations would be useful in the treatment of HCV in patients who are also being treated with drugs that modulate gastric pH (e.g., proton pump inhibitors).

The need for formulations to effectively promote oral drug absorption and for formulations that provide increased absorption and/or enhanced insensitivity to variations in gastric pH continues to grow, but their design remains largely unpredictable. Such formulations of drug substances may provide effective absorption following oral administration, which is useful to reduce pill burden (e.g., the number of tablets administered), regimen complexity (e.g., eliminating the need to administer with food or without food), and facilitate co-dosing with other medications, such as antacid medications. Formulations with this type of enhanced absorption will ultimately improve compliance, and, therefore, efficacy.

The current invention relates to novel formulations of Compound A, which may provide improved oral absorption, confer insensitivity to higher gastric pH, enhance dissolution rate, and/or maintain higher supersaturation of Compound A relative other formulations.

### SUMMARY OF THE INVENTION

The present disclosure relates to tablets comprising pharmaceutical formulations comprising dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, Compound A:

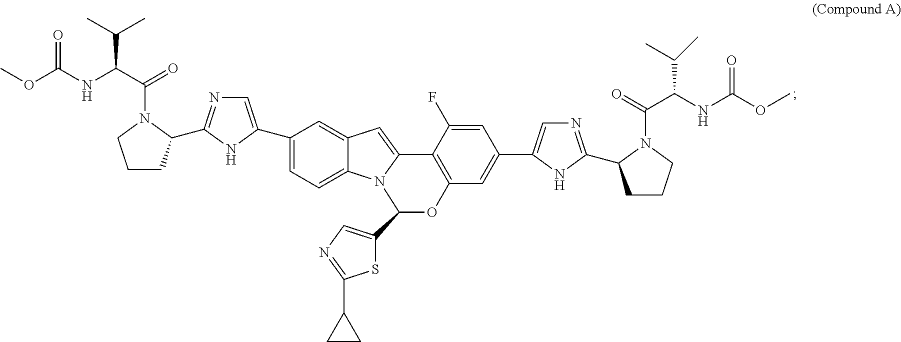

wherein Compound A is substantially amorphous. Embodiments of such formulations may be prepared by wet granulation or by roller compaction. Embodiments may additionally comprise diluents, disintegrants, lubricants, salts, glidants, binders, surfactants, solubilizers, wetting agents, and/or fillers. In embodiments, formulations of the disclosure may provide improved oral bioavailability and/or insensitivity to gastric pH.

Other embodiments, aspects and features of the present invention are either further described in or will be apparent from the ensuing description, examples, and claims.

### DETAILED DESCRIPTION OF THE INVENTION

Compound A is a weak base, with two basic sites, which protonate at low pH giving rise to pH-dependent solubility profile. This pH-dependent solubility could significantly impair the amount of Compound A dissolved from formulations in patients with elevated gastric pH, which in turn could lead to potentially lower absorption. In order to mitigate variability in absorption of Compound A due to elevated gastric pH, solid dispersion formulations of Compound A may be formulated at a drug loading up to approximately 45% in combination with pharmaceutically suitable polymers and surfactants.

Compound A may be in the form of a pharmaceutically acceptable salt. In additional instances, Compound A may also be anhydrous or in the form of a hydrate or solvate.

In the present pharmaceutical formulations, Compound A is provided in a form that is substantially amorphous. The substantially amorphous Compound A may be formulated directly as provided from synthetic preparation or it may be spray-dried to provide substantially amorphous Compound A. In particular, the substantially amorphous Compound A may be spray dried with a solvent, with a solvent and a surfactant, with a solvent and a pharmaceutically acceptable polymer, or with a solvent, a surfactant, and a pharmaceutically acceptable polymer. Thus, substantially amorphous Compound A may be provided directly from synthesis, as spray-dried compound, as a spray-dried composition comprising Compound A and a surfactant, as a spray-dried composition comprising Compound A and a pharmaceutically acceptable polymer, or as a spray-dried composition comprising Compound A, a surfactant, and a pharmaceutically acceptable polymer. As demonstrated by the Examples, pharmaceutical formulations comprising spray-dried compositions including Compound A showed robust pharmacokinetic performance when dosed with pH-raising medication. When a formulation containing a spray-dried composition of Compound A was prepared as an oral dosage form as described herein, it was found to maintain the pharmacokinetic performance of Compound A and to provide robust absorption regardless of gastric pH modulation due to the use of, for example, H2-receptor antagonists or proton-pump inhibitors.

Compound A, either directly from synthesis or in a spray-dried composition, may be directly formulated as a solid dosage form by blending or granulating with excipients and compressed into tablets or filled into hard capsule shells. The granulation process may be a wet granulation process, such as high-shear wet granulation or fluidized-bed granulation, or it may be a dry granulation process, such as roller-compaction.

Wet granulation is defined as a process involving granulating the powder with liquid (aqueous, non-aqueous, hot melt, etc.) to achieve the desired properties for subsequent downstream processes. The liquid (e.g., water, binder solution) is added to the powder blend while the powder blend is being continuously mixing, leading to granule nucleation and growth. When the formulation is to be processed by wet granulation, substantially amorphous Compound A may be provided directly, “as is”, from synthesis or as spray-dried compound (spray dried from Compound A and a solvent).

Roller compaction is defined as an agglomeration process where a powder is compressed into a dense compact (or ribbon) using two counter-rotating rollers. The pressing forces from the rollers cause the particles to deform plastically to achieve the dense compact. The dense compact is subsequently broken up and milled into granules. The advantages of roller compaction include improved flow, better content uniformity, and/or less sticking. Roller compaction is also preferred for moisture sensitive and/or heat sensitive formulations. When the formulation is to be processed by roller compaction, substantially amorphous Compound A may be provided as spray-dried intermediate and a pharmaceutically acceptable polymer, or as spray-dried intermediate, a surfactant, and a pharmaceutically acceptable polymer.

The pharmaceutical formulations may be roller compacted or wet granulated to densify and/or reduce the risk of segregation of components during subsequent handling (e.g., compression into tablets). Granulation steps can also be used to minimize the impact of raw material property variability (e.g., excipient particle size) on subsequent processing (e.g., tablet compression) and ultimate product performance. Lubrication is typically performed prior to roller compaction and tablet compression to reduce the tendency of material to adhere to compression surfaces (e.g., tablet tooling). In particular embodiments, the lubricant system is a combination of sodium stearyl fumarate and magnesium stearate. These methods can be carried out by those skilled in the art. See, e.g., Ansel, Introduction to Pharmaceutical Dosage Forms, Seventh Edition, 1999.

Tablets may contain the active ingredient in admixture with non-toxic pharmaceutically acceptable excipients that are suitable for the manufacture of tablets. These excipients may be for example, inert diluents, granulating and disintegrating agents, binding agents, glidants, lubricating agents, and antioxidants, for example, propyl gallate, butylated hydroxyanisole, and butylated hydroxy toluene. The tablets may be uncoated or they may be film coated to modify their appearance or may be coated so as to modulate the onset and/or rate of release in the gastrointestinal tract, so as to optimize or maximize the biological exposure of the patient to Compound A. In embodiments, the tablets are coated with a functional coat to delay disintegration and absorption in the gastrointestinal tract and thereby provide a sustained action over a longer period.

To prepare the pharmaceutical compositions of the invention, the pharmaceutical formulation is compressed into an oral dosage form such as tablets. Tablets can be prepared with a variety of possible shapes (ellipsoidal, capsule, biconvex round, etc.). Techniques suitable for preparing solid oral dosage forms of the present invention are described in Remington's Pharmaceutical Sciences, 18th edition, edited by A. R. Gennaro, 1990, Chapter 89 and in Remington—The Science and Practice of Pharmacy, 21st edition, 2005, Chapter 45.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents.

As used herein, the term “amorphous” indicates that the material lacks a high degree of order on a molecular level and may exhibit the physical properties of a solid or a liquid, depending on the temperature of the material. Amorphous materials do not give X-ray diffraction patterns with distinctive sharp peaks.

As used herein, the term “crystalline” indicates that the material has a regular ordered internal structure at the molecular level when in the solid phase, and the crystalline material gives a distinctive X-ray diffraction pattern with defined peaks.

As used herein, the term “substantially amorphous” refers to a composition in which greater than 70%; or greater than 75%; or greater than 80%; or greater than 85%; or greater than 90%; or greater than 95%, or greater than 99% of the Compound A is amorphous. “Substantially amorphous” can also refer to material that has no more than about 20% crystallinity, or no more than about 10% crystallinity, or no more than about 5% crystallinity, or no more than about 2% crystallinity.

As used herein, the term “substantially crystalline” refers to a composition in which greater than 70%; or greater than 75%; or greater than 80%; or greater than 85%; or greater than 90%; or greater than 95%, or greater than 99% of the Compound A is crystalline. “Substantially crystalline” can also refer to material that has no more than about 20% crystallinity, or no more than about 10% amorphous, or no more than about 5% amorphous, or no more than about 2% amorphous.

The term “effective amount” indicates a sufficient amount to exert a therapeutic or prophylactic effect. For a patient who is infected with HCV, an effective amount is sufficient to achieve one or more of the following effects: reduce the ability of HCV to replicate, reduce HCV load, and increase viral clearance. For a patient who is not infected with HCV, an effective amount is sufficient to achieve one or more of a reduced susceptibility to HCV infection, and a reduced ability of the infecting virus to establish persistent infection for chronic disease.

The term “subject” (alternatively referred to herein as “patient”) as used herein refers to an animal, preferably a mammal, most preferably a human, who has been the object of treatment, observation or experiment.

Compound A, as provided in the formulations and/or the oral dosage forms described herein, independently may take the form of pharmaceutically acceptable salts. The term “pharmaceutically acceptable salt” refers to a salt of the parent compound that has activity and that is not biologically or otherwise undesirable (e.g., is neither toxic nor otherwise deleterious to the recipient thereof); also included in this term are complexes that comprise solvent molecules and a salt of the parent compound. Suitable salts include acid addition salts that may, for example, be formed by mixing a solution of a compound with a solution of a pharmaceutically acceptable acid such as hydrochloric acid, sulfuric acid, acetic acid, trifluoroacetic acid, benzoic acid, phosphoric acid, methanesulfonic acid, naphthalene-1,5-disulfonic acid, and toluenesulfonic acid. In instances, Compound A may be in the form of bis-tosylate salt of Compound A.

The term “polymer” as used herein refers to a chemical compound or mixture of compounds consisting of repeating structural units created through a process of polymerization. Suitable polymers useful in this invention are described throughout. When specific polymers that are suitable for use in the compositions of the present invention are blended, the blends of such polymers may also be suitable. Thus, the term “polymer” is intended to include blends of polymers in addition to a single species of polymer.

In the embodiments described herein, any variable or component is as defined in the first instance where the variable or component occurs, unless otherwise indicated. When any variable or component occurs more than one time, its selection on each occurrence is independent of its selection at every other occurrence, unless it is expressly stated otherwise. Also, combinations of embodiments, variables or components are permissible only if such combinations result in stable formulations, blends, or oral dosage forms.

### Tablets Comprising Pharmaceutical Formulations of Compound A Prepared by Roller Compaction

A first embodiment relates to tablets comprising pharmaceutical formulations comprising dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, Compound A:

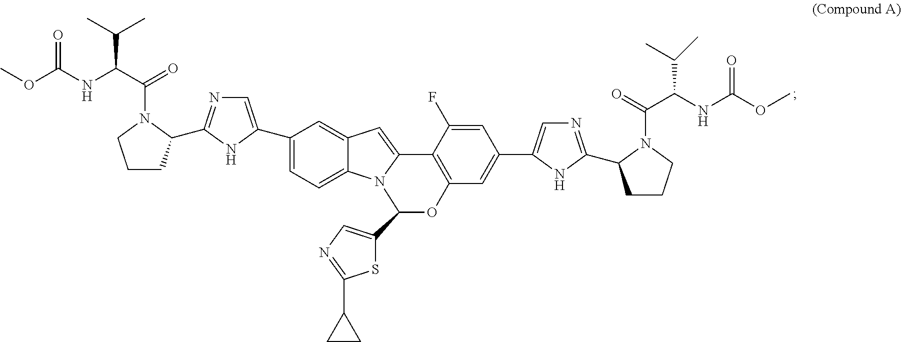

wherein Compound A is amorphous, and said formulation is prepared by roller compaction.

In aspects of the first embodiment, Compound A is spray-dried to provide amorphous Compound A. In particular, the amorphous Compound A may be spray dried with a solvent and a pharmaceutically acceptable surfactant, with a solvent and a pharmaceutically acceptable polymer, or with a solvent, a pharmaceutically acceptable surfactant, and a pharmaceutically acceptable polymer. Thus, amorphous Compound A may be provided as a spray-dried composition comprising Compound A and a pharmaceutically acceptable surfactant, as a spray-dried composition comprising Compound A and a pharmaceutically acceptable polymer, or as a spray-dried composition comprising Compound A, a pharmaceutically acceptable surfactant, and a pharmaceutically acceptable polymer.

Compound A is present in the spray-dried composition in a total concentration of from about 5% w/w to about 50% w/w. In particular instances, Compound A is present in a total concentration of from about 10% w/w to about 40% w/w, or about 20% w/w. All other variables are as provided above.

The pharmaceutically acceptable polymers may enhance the absorption of Compound A when used in the spray-dried compositions described herein. The pharmaceutically acceptable polymers are selected from the group consisting of cellulosic polymers and vinyl pyrrolidone/vinyl acetate copolymers.

Cellulosic polymers include cellulose esters or cellulose ethers, such as alkylcelluloses (e.g., methylcellulose or ethylcellulose), hydroxyalkyl celluloses (e.g., hydroxypropyl cellulose), hydroxyalkyl alkylcelluloses (e.g., hydroxypropyl methylcellulose), and cellulose phthalates or succinates (e.g., hydroxypropyl methylcellulose phthalate, cellulose acetate phthalate, hydroxypropyl methylcellulose succinate, or hydroxypropyl methylcellulose acetate succinate (HPMCAS)). Commercially available examples of these include hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P 55.

The pharmaceutically acceptable polymer may be vinyl pyrrolidone/vinyl acetate copolymers. In particular instances, the pharmaceutically acceptable polymer is copovidone, a copolymer of 1-vinyl-2-pyrrolidone and vinyl acetate in the mass proportion of 3:2. Other useful copolymers contain vinyl pyrrolidone and vinyl acetate in ratios of, for example, 90:10, 80:20, 70:30, and 50:50. The amount of vinyl pyrrolidone can range from about 40% up to about 99.9%, and the amount of vinyl acetate can range from about 0.1% up to about 60%. Other vinyl polymers and copolymers having substituents that are hydroxy, alkyl, acyloxy, or cyclic amides include polyethylene polyvinyl alcohol copolymers; and polyvinyl caprolactam-polyvinyl acetate-polyethylene glycol graft copolymer (SOLUPLUS®, BASF Corp.). Commercially available copolymers of vinyl pyrrolidone and vinyl acetate include PLASDONE® S630 (Ashland, Inc., Covonton, KY) and KOLLIDON® VA 64 (BASF Corp., Florham Park, NJ), which contain vinyl pyrrolidone and vinyl acetate in a 60:40 ratio. Other copolymers of vinyl pyrrolidone and vinyl acetate can also be used in the invention. Preferably, the copolymer contains at least 40% vinyl pyrrolidone, although smaller amounts of vinyl pyrrolidone can also be utilized.

The pharmaceutically acceptable polymer may be non-ionic.

The pharmaceutically acceptable polymers are selected from the group consisting of cellulosic polymers and vinyl pyrrolidone/vinyl acetate copolymers. In particular aspects of this embodiment, the pharmaceutically acceptable polymer is selected from the group consisting of HPMC, HPMCAS and hydroyxpropyl methylcellulose phthalate (HPMCP). In particular instances, the pharmaceutically acceptable polymer is HPMC. In aspects of these instances, the HPMC is present in an amount in a range from about 40% up to about 99.9%. In other particular instances, the pharmaceutically acceptable polymer is HPMCAS. In aspects of these instances, the HPMCAS is present in an amount in a range from about 40% up to about 99.9%.

The pharmaceutically acceptable polymers are present in a total concentration of from about 50% w/w to about 95% w/w. In instances, the pharmaceutically acceptable polymers are present in a total concentration of from about 50% w/w to about 90% w/w, or about 70% w/w.

Surfactants can increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Surfactants may also facilitate absorption of drugs by altering drug permeability and/or efflux directly. See, e.g., Yu et al., 16 PHARM RES. 1812-1817 (1999). Non-limiting examples of pharmaceutically acceptable surfactants that are suitable for the present invention include polyoxyethylene castor oil derivates, e.g. polyoxyethyleneglycerol triricinoleate or polyoxyl 35 castor oil (CREMOPHOR® EL; BASF Corp.) or polyoxyethylene glycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or polysorbates or mono fatty acid esters of polyoxyethylene sorbitan, such as a mono fatty acid ester of polyoxyethylene (20) sorbitan, e.g. polyoxyethylene (20) sorbitan monooleate (commercially available as TWEEN® 80), polyoxyethylene (20) sorbitan monostearate (commercially available as TWEEN® 60), polyoxyethylene (20) sorbitan monopalmitate (commercially available as TWEEN® 40), or polyoxyethylene (20) sorbitan monolaurate (commercially available as TWEEN® 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate, sucrose palmitate; sorbitan fatty acid mono esters such as sorbitan mono laurate (commercially available as SPAN® 20), sorbitan monooleate (commercially available as SPAN® 80), sorbitan monopalmitate (commercially available as SPAN® 40), or sorbitan stearate; D-alpha-tocopheryl polyethylene glycol 1000 succinate (TPGS); or a combination or mixture thereof. Other non-limiting examples of suitable surfactants include anionic surfactants, e.g. docusate potassium, docusate sodium, docusate calcium, and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as POLOXAMER® 124, POLOXAMER® 188, POLOXAMER® 237, POLOXAMER® 388, or POLOXAMER® 407 (BASF Corp.). As described above, a mixture of surfactants can be used in a spray-dried composition as described herein. In particular instances, the surfactant is selected from the group consisting of sodium lauryl sulfate (SLS), D-α-tocopheryl polyethylene glycol 1000 succinate (TPGS), or nonionic ethoxylated alcohols like polysorbate or poloxamer. In aspects of this spray-dried composition, the surfactant may be selected from the group consisting of sodium lauryl sulfate (SLS), sucrose palmitate, D-α-tocopheryl polyethylene glycol 1000 succinate (TPGS), or nonionic ethoxylated alcohols like polysorbate or poloxamer. In particular instances, the pharmaceutically acceptable surfactant is sucrose palmitate, TPGS, or a combination thereof.

The pharmaceutically acceptable surfactant may be present in a concentration of from about 2% w/w to about 20% w/w. In particular instances, the pharmaceutically acceptable surfactant is present in a concentration of from about 1% w/w to about 15% w/w, or from about 1% w/w to about 10% w/w, or from about 5% w/w to about 8% w/w.

The spray-dried composition may be in the form of particles.

The spray-dried composition described herein are prepared by processes that are suitable for causing Compound A to form an amorphous dispersion, such that Compound A is generally amorphous or dissolved in a component of the composition, such as a polymer and/or a surfactant. The dispersions are stable, and the drug does not form crystals or other insoluble particles. Spray drying is well known (see, e.g., Masters, Spray Drying Handbook, 1991, 5^{th} edition, Longman Scientific & Technical) and widely practiced in a variety of industrial applications including spray drying of milk (see, e.g., U.S. Pat. No. 4,187,617) and pharmaceutical products (see, e.g., U.S. Pat. No. 6,763,607). To produce spray-dried compositions, the drug, optional polymer, and optional surfactant, are dissolved in a solvent and then are sprayed through a nozzle as a fine spray into a chamber, where the solvent is evaporated quickly to make particles of a composition comprising drug, optional polymer, and optional surfactant. Ideally, the solvent is any solvent in which all of the components of the composition are soluble and that is readily evaporated in a spray dryer. The solvent should also be suitable for use in preparing pharmaceutical compositions. The use of mixed-solvent systems, particularly those containing a combination of water and another solvent, may be necessary to facilitate the production of spray-dried compositions containing Compound A, an optional polymer or polymer(s), and, optionally a surfactant.

Useful solvents for spray drying include water, acetone, ethanol, methanol, dichloromethane, isopropanol, and tetrahydrofuran (THF). In aspects, the mixed-solvent system consists of a first solvent and a second solvent, in which the first solvent may be selected from the group consisting of acetone, ethanol, methanol, dichloromethane, isopropanol, and THF; the second solvent is water. In particular aspects, the first solvent may be selected from the group consisting of ethanol, methanol, and acetone; the second solvent is water. In specific instances, the first solvent is acetone, and the second solvent is water. The proportions of the first solvent to second solvent may be about 90:10, about 80:20, about 70:30, or about 60:40. Mixed-solvent systems are described in International Patent Application Publication No. WO2007/109605 and U.S. Patent Application Publication No. US2007/0026083. Solids loading, which usually refers to the concentration of solid components in the spray drying solvent system, does not typically exceed 50% and depends on solution properties, such as solubility, stability, and viscosity. The solids, comprising Compound A, the optional polymer, and optional surfactant, are present in the spray drying solution in a total concentration of from about 5% w/w to about 25% w/w, based on the solubility, stability, and viscosity of the solution. In particular instances, the solids are present in the solution in a total concentration of from about 10% w/w to about 20% w/w.

Following formation of a spray-dried composition, a secondary drying step may be employed to remove residual solvents, which may occur in a static dryer or agitated dryer. Gas, humidified gas, or vacuum may be applied to the material in the secondary dryer and such application can be useful in more rapidly removing residual solvents that remain in the spray-dried composition. See, e.g., European Patent Application No. EP1855652 A2 (and references therein) and International Patent Application Publication No. WO2008/012617A1 (and references therein).

As demonstrated by the Examples, the oral absorption of Compound A when formulated with one or more polymer, such as HPMC, together with optional surfactants, such as TPGS, as a spray-dried composition, is superior to formulations based on undispersed amorphous Compound A.

The relative amount of drug, optional polymer, and optional surfactant can vary widely. The optimal amounts of the polymer and surfactant can depend, for example, the hydrophilic lipophilic balance (HLB), melting point, and water solubility of the copolymer, and the surface tension of aqueous solutions of the surfactant, the properties of the drug, etc.

The spray-dried compositions comprise an effective amount of Compound A, but comprise less than about 50% w/w of Compound A due to the relatively poor dissolution seen with formulations having greater than 50% w/w of Compound A. Thus, the concentration of Compound A can vary from about 0.1% to about 40.0%, from about 5.0% to about 35.0%, or from about 10% to about 30%, by weight based on the total combined weight of Compound A, optional polymer, and optional surfactant (not including other excipients).

The concentration of the pharmaceutically acceptable surfactant, when present, in the spray-dried composition can vary from about 2.0% to about 20%, or about 5% to about 15%, or about 10% by weight based on the total combined weight of Compound A, optional polymer, and optional surfactant (not including other excipients).

The concentration of the pharmaceutically acceptable polymer, when present, in the spray-dried composition is added to the concentrations of the Compound A and surfactant to add up to 100%. The concentration can vary from about 50% to about 95% by weight based on the total combined weight of Compound A, optional polymer, and optional surfactant, not including other excipients.

The spray-dried composition may comprise from 5% to 50% of Compound A or a pharmaceutically acceptable salt thereof, 2.0% to about 20% surfactant, with the balance of the formulation being the polymer.

Thus, the first embodiment provides pharmaceutical formulations comprising spray-dried compositions comprising Compound A, as described above. In aspects of the first embodiment, spray-dried compositions comprising Compound A is present in the pharmaceutical formulation in a total concentration of from about 3% w/w to about 45% w/w. In particular instances, Compound A is present in the pharmaceutical formulation in a total concentration of from about 15% w/w to about 25% w/w, or about 30% w/w.

In a first aspect of the first embodiment, the pharmaceutical formulation includes a pharmaceutically acceptable diluent selected from the group consisting of mannitol, microcrystalline cellulose, calcium carbonate, sodium carbonate, lactose, dicalcium phosphate, sodium phosphate, and starch, and combinations thereof. In particular aspects, the diluent is selected from the group consisting of microcrystalline cellulose, mannitol, and dicalcium phosphate. In a particular instance, the diluent is a combination of mannitol and microcrystalline cellulose. The diluent is present in the pharmaceutical formulation in a total concentration of from about 3% w/w to about 50% w/w. In particular instances, the diluent is present in a total concentration of from about 18% w/w to about 55% w/w, or about 50% w/w.

In a second aspect of the first embodiment, the pharmaceutical formulation includes a pharmaceutically acceptable disintegrant. Disintegrants can be used in the formulations to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical formulations comprise from about 0.5 to about 15 weight percent of disintegrant, specifically from about 1 to about 5 weight percent of disintegrant. Disintegrants that can be used in the pharmaceutical formulations provided herein include, but are not limited to, croscarmellose sodium, crospovidone, sodium starch glycolate, potato or tapioca starch, pre-gelatinized starch, other starches, other celluloses, gums, and mixtures thereof. The disintegrant in the pharmaceutical formulation of embodiments may be selected from the group consisting of croscarmellose sodium, sodium starch glycolate, and crospovidone. In particular instances, the disintegrant is croscarmellose sodium. The disintegrant may be present in the pharmaceutical formulation in a total concentration of from about 4% w/w to about 20% w/w. In particular instances, the disintegrant is present in a total concentration of from about 7% w/w to about 15% w/w, or about 9% w/w.

In a third aspect of the first embodiment, the pharmaceutical formulation includes a pharmaceutically acceptable lubricant. Lubricants that can be used in the pharmaceutical formulations provided herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Lubricants are typically used in an amount of less than about 1 weight percent of the pharmaceutical formulations or dosage forms into which they are incorporated. The lubricant in the pharmaceutical formulation may be pharmaceutically acceptable diluents selected from the group consisting of magnesium stearate, sodium stearyl fumarate, stearic acid, and glyceryl behenate. In a particular instance, the lubricant is a combination of magnesium stearate and sodium stearyl fumarate. In particular instances, the lubricant is present in the pharmaceutical formulation in a total concentration of from about 0.5% w/w to about 4% w/w. In particular, the lubricant is present in a total concentration of from about 1% w/w to about 3% w/w, or about 2% w/w.

In a fourth aspect of the first embodiment, an ionic salt may be present in the pharmaceutical formulation to further enhance the disintegration of the dosage form. The salt is selected from the group consisting of NaCl, KCl, CaCl₂, KH₂PO₄, NaH₂PO₄, K₂SO₄, NaHCO₃, K₂CO₃, and combinations thereof. In aspects, the salt in the pharmaceutical formulation is selected from the group consisting of NaCl, KCl, and CaCl₂, and combinations thereof. In a particular instance, the salt is NaCl. The salt may be present in the pharmaceutical formulation in a total concentration of from about 0% w/w to about 30% w/w. In particular instances, the salt is present in a total concentration of from about 7% w/w to about 18% w/w, or about 10% w/w.

In a fifth aspect of the first embodiment, a glidant may be incorporated into the pharmaceutical formulation. The glidant in the pharmaceutical formulation may be selected from the group consisting of starch, talc, magnesium stearate, and silicon dioxide, and combinations thereof. In a particular instance, the glidant is silicon dioxide. The glidant may be present in the blended material in a total concentration of from about 0% w/w to about 2% w/w. In particular instances, the glidant is present in a total concentration of from about 0.1% w/w to about 1% w/w, or about 0.25% w/w.

In a sixth aspect of the first embodiment, a binder and/or a filler may be incorporated into the pharmaceutical formulation. Binders suitable for use in the pharmaceutical compositions provided herein include, but are not limited to, starches, cellulose and its derivatives (e.g., ethylcellulose, cellulose acetate, carboxymethyl cellulose calcium, sodium carboxymethyl cellulose, methylcellulose, hydroxypropyl methylcellulose, hydroxypropyl cellulose), polyvinyl pyrrolidone, and mixtures thereof. Examples of fillers suitable for use in the pharmaceutical compositions provided herein include, but are not limited to, microcrystalline cellulose, powdered cellulose, mannitol, lactose, calcium phosphate, starch, pre-gelatinized starch, and mixtures thereof. The binder in pharmaceutical compositions is typically present in from about 50 to about 99 weight percent of the pharmaceutical composition or dosage form.

In a seventh aspect of the first embodiment, a lubricant system is included in the pharmaceutical formulation, such as a combination of sodium stearyl fumarate and magnesium stearate.

Pharmaceutical compositions intended for oral use, such as those of the first embodiment, may further contain agents selected from the group consisting of sweetening agents, flavoring agents, coloring agents, and preserving agents in order to provide pharmaceutically elegant and palatable preparations.

### Tablets Comprising Pharmaceutical Formulations of Compound A Prepared by Wet Granulation

A second embodiment relates to tablets comprising pharmaceutical formulations comprising dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, Compound A:

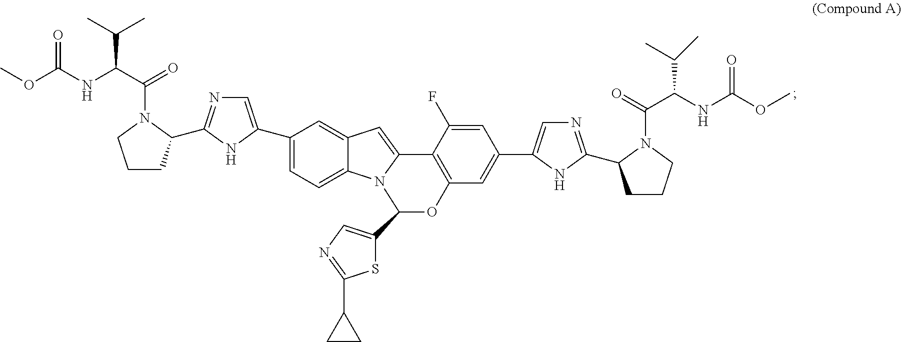

wherein Compound A is amorphous, and said formulation is prepared by wet-granulation.

In aspects of the second embodiment, Compound A may be formulated directly as provided from synthesis, or Compound A may be spray-dried, as discussed above, with a solvent to provide amorphous compound. Compound A is present in the pharmaceutical formulation in a total concentration of from about 3% w/w to about 45% w/w. In particular instances, Compound A is present in the pharmaceutical formulation in a total concentration of from about 15% w/w to about 35% w/w, or about 30% w/w.

In a first aspect of the second embodiment, the pharmaceutical formulation includes a pharmaceutically acceptable diluent selected from the group consisting of mannitol, microcrystalline cellulose, calcium carbonate, sodium carbonate, lactose, dicalcium phosphate, sodium phosphate, and starch, and combinations thereof. In particular aspects, the diluent is selected from the group consisting of microcrystalline cellulose, mannitol, and dicalcium phosphate. In a particular instance, the diluent is a combination of mannitol and microcrystalline cellulose. The diluent is present in the pharmaceutical formulation in a total concentration of from about 3% w/w to about 50% w/w. In particular instances, the diluent is present in a total concentration of from about 18% w/w to about 55% w/w, or about 50% w/w.

In a second aspect of the second embodiment, the pharmaceutical formulation includes a pharmaceutically acceptable disintegrant. Disintegrants that can be used in the pharmaceutical formulations of the second embodiment include, but are not limited to, croscarmellose sodium, crospovidone, sodium starch glycolate, potato or tapioca starch, pre-gelatinized starch, other starches, other celluloses, gums, and mixtures thereof. The disintegrant in the pharmaceutical formulation of embodiments may be selected from the group consisting of croscarmellose sodium, sodium starch glycolate, and crospovidone. In particular instances, the disintegrant is croscarmellose sodium. The disintegrant may be present in the pharmaceutical formulation in a total concentration of from about 4% w/w to about 20% w/w. In particular instances, the disintegrant is present in a total concentration of from about 7% w/w to about 15% w/w, or about 9% w/w.

In a third aspect of the second embodiment, the pharmaceutical formulation includes a pharmaceutically acceptable lubricant. Lubricants that can be used in the pharmaceutical formulations of the second embodiment include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. The lubricant in the pharmaceutical formulation may be selected from the group consisting of magnesium stearate, sodium stearyl fumarate, stearic acid, and glyceryl behenate. In a particular instance, the lubricant is a combination of magnesium stearate and sodium stearyl fumarate. In particular instances, the lubricant is present in the pharmaceutical formulation in a total concentration of from about 0.5% w/w to about 4% w/w. In particular, the lubricant is present in a total concentration of from about 1% w/w to about 3% w/w, or about 2% w/w.

In a fourth aspect of the second embodiment, an ionic salt may be present in the pharmaceutical formulation to further enhance the disintegration of the dosage form. The salt is selected from the group consisting of NaCl, KCl, CaCl₂, KH₂PO₄, NaH₂PO₄, K₂SO₄, NaHCO₃, K₂CO₃, and combinations thereof. In aspects, the salt in the pharmaceutical formulation is selected from the group consisting of NaCl, KCl, and CaCl₂, and combinations thereof. In a particular instance, the salt is NaCl. The salt may be present in the pharmaceutical formulation in a total concentration of from about 0% w/w to about 30% w/w. In particular instances, the is present in a total concentration of from about 7% w/w to about 18% w/w, or about 10% w/w.

In a fifth aspect of the second embodiment, a glidant may be incorporated into the pharmaceutical formulation. The glidant in the pharmaceutical formulation may be selected from the group consisting of starch, talc, magnesium stearate, and silicon dioxide, and combinations thereof. In a particular instance, the glidant is silicon dioxide. The glidant may be present in the blended material in a total concentration of from about 0% w/w to about 2% w/w. In particular instances, the glidant is present in a total concentration of from about 0.1% w/w to about 1% w/w, or about 0.25% w/w.

In a sixth aspect of the second embodiment, a binder and/or filler may be incorporated into the pharmaceutical formulation. Binders suitable for use in the pharmaceutical formulations provided herein include, but are not limited to, starches, cellulose and its derivatives (e.g., ethylcellulose, cellulose acetate, carboxymethyl cellulose calcium, sodium carboxymethyl cellulose, methylcellulose, hydroxypropyl methylcellulose, hydroxypropyl cellulose), polyvinyl pyrrolidone, and mixtures thereof. Examples of fillers suitable for use in the pharmaceutical formulations provided herein include, but are not limited to, microcrystalline cellulose, powdered cellulose, mannitol, lactose, calcium phosphate, starch, pre-gelatinized starch, and mixtures thereof. The binder in pharmaceutical formulations is typically present in from about 50 to about 99 weight percent of the pharmaceutical formulations or dosage form.

In a seventh aspect of the second embodiment, a solubilizer may be present in the pharmaceutical formulation, to increase the solubility of Compound A. The solubilizer may be selected from pharmaceutically acceptable surfactants. Non-limiting examples of pharmaceutically acceptable surfactants that are suitable for use as solubilizers include polyoxyethylene castor oil derivates, e.g. polyoxyethyleneglycerol triricinoleate or polyoxyl 35 castor oil (CREMOPHOR® EL; BASF Corp.) or polyoxyethyleneglycerol oxystearate such as polyethylenglycol 40 hydrogenated castor oil (CREMOPHOR® RH 40, also known as polyoxyl 40 hydrogenated castor oil or macrogolglycerol hydroxystearate) or polyethylenglycol 60 hydrogenated castor oil (CREMOPHOR® RH 60); or polysorbates or mono fatty acid esters of polyoxyethylene sorbitan, such as a mono fatty acid ester of polyoxyethylene (20) sorbitan, e.g. polyoxyethylene (20) sorbitan monooleate (commercially available as TWEEN® 80), polyoxyethylene (20) sorbitan monostearate (commercially available as TWEEN® 60), polyoxyethylene (20) sorbitan monopalmitate (commercially available as TWEEN® 40), or polyoxyethylene (20) sorbitan monolaurate (commercially available as TWEEN® 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); sucrose fatty acid esters, e.g. sucrose monostearate, sucrose distearate, sucrose monolaurate, sucrose dilaurate, sucrose palmitate; sorbitan fatty acid mono esters such as sorbitan mono laurate (commercially available as SPAN® 20), sorbitan monooleate (commercially available as SPAN® 80), sorbitan monopalmitate (commercially available as SPAN® 40), or sorbitan stearate; D-alpha-tocopheryl polyethylene glycol 1000 succinate (TPGS); or a combination or mixture thereof. Other non-limiting examples of suitable surfactants include anionic surfactants, e.g. docusate potassium, docusate sodium, docusate calcium, and sodium lauryl sulfate (SLS). Other suitable surfactants include, but are not limited to, block copolymers of ethylene oxide and propylene oxide, also known as polyoxyethylene polyoxypropylene block copolymers or polyoxyethylene polypropyleneglycol, such as POLOXAMER® 124, POLOXAMER® 188, POLOXAMER® 237, POLOXAMER® 388, or POLOXAMER® 407 (BASF Corp.). In particular instances, the solubilizer is D-α-tocopheryl polyethylene glycol 1000 succinate (TPGS). The solubilizer may be present in a concentration of from about 2% w/w to about 15% w/w. In particular instances, the pharmaceutically acceptable solubilizer is present in a concentration of about 8% w/w.

In an eighth aspect of the second embodiment, a wetting agent may be present in the pharmaceutical formulation, to increase the solubility of Compound A. The wetting agent may be selected from pharmaceutically acceptable surfactants; surfactants listed above as solublilzers may also be suitable as wetting agents. In further particular instances, the wetting agent is sucrose palmitate. The wetting agent may be present in a concentration of from about 1% w/w to about 10% w/w. In particular instances, the pharmaceutically acceptable wetting agent is present in a concentration of about 5% w/w.

As discussed above in relation to the first embodiment, pharmaceutical formulations intended for oral use, such as those of the second embodiment, may further contain agents selected from the group consisting of sweetening agents, flavoring agents, coloring agents, and preserving agents in order to provide pharmaceutically elegant and palatable preparations.

A third embodiment is directed to a process for preparing a blended material by i) blending amorphous Compound A with a diluent, disintegrant, lubricant, salt, glidant, solubilizer, wetting agent, binder, and/or filler, and ii) optionally granulating, to produce the blended material. Granulation, as used herein, includes all known and later-developed methods of creating granules. The diluent, disintegrant, lubricant, salt, glidant, solubilizer, wetting agent, binder, and/or filler are as described above with respect to the first and second embodiments.

A fourth embodiment is directed to a process for preparing a solid pharmaceutical composition comprising the steps of: a) preparing a blended material as described above in the third embodiment; b) compressing the blended material into a tablet. In aspects of the fourth embodiment, the tablet is optionally film-coated; in further aspects, the tablet is optionally photo-shielded, for example by use of a blister packaging.

The following examples serve only to illustrate the invention and its practice. The examples are not to be construed as limitations on the scope or spirit of the invention.

In addition, the following abbreviations are used throughout this specification and in the Examples. Each of these terms has the meaning listed below.

### Abbreviations

AUC_{0-∞} Area under the concentration time curve from time zero to infinity
AUC₀₋ₗₐₛₜ Area under the concentration time curve from time zero to last dose
AUC₀₋₂₄ Area under the concentration time curve from time zero to 24 hours
bar Metric unit of pressure, 1 bar=100,000 Pascal
CI Confidence interval
Cₘₐₓ Maximum concentration (specifically of a drug)
C₂₄ Maximum concentration over 24 hours (specifically of a drug)
GM Geometric mean
GMR Geometric mean ratio
HPMC Hydroxypropylmethyl cellulose
HPMCAS Hydroxypropylmethyl cellulose acetate succinate
kP, kgf Kilopond, a non-standard gravitational unit of force, also kilogram-force; 1 kP=9.80665 Newtons
PSI, psi Pounds per square inch [gauge], 1 Pascal=0.000145037738007 psi
RH Relative Humidity
RPM Revolutions per minute
SLS Sodium lauryl sulfate
TPGS Vitamin E polyethylene glycol succinate
w/w, % w/w Percentage by weight (i.e., g of solute in 100 g of solution), weight percent

### EXAMPLES

### Example 1: Roller-Compacted Formulation of Compound A

Formulation 1 is a roller-compacted tablet formulation in which Compound A is formulated as a pure amorphous Compound A (Table 1). Compound A was blended with microcrystalline cellulose, mannitol, sucrose palmitate, and two-thirds of the croscarmellose sodium and lubricated with half of the magnesium stearate. The resulting blends are compacted into slugs. The slugs were milled through a screen with an opening size of approximately 1 mm. The resulting granules are blended with the remaining croscarmellose sodium, lubricated with the magnesium stearate, and compressed into tablets. The compression parameters were adjusted to achieve acceptable hardness, friability, and disintegration time.

TABLE 1
Composition of Formulation 1

| | Formulation 1 |
|---|---|
| Component | Amount (mg/tablet) |
| Compound A | 60.0 |
| Microcrystalline cellulose | 55.0 |
| Mannitol | 55.0 |
| Croscarmellose sodium | 18.0 |
| Sucrose palmitate | 10.0 |
| Magnesium stearate | 2.0 |
| Total | 200.0 |

### Example 2: Roller-Compacted Formulations of Compound A

Formulations 2 and 3 are roller-compacted tablet formulation in which Compound A is formulated as a pure amorphous API (Table 2). Compound A was blended with microcrystalline cellulose, mannitol, sucrose palmitate, or poloxamer, and two-thirds of the croscarmellose sodium and lubricated with half of the magnesium stearate. The resulting blends are compacted into slugs. The slugs were milled through a screen with an opening size of approximately 1 mm. The resulting granules are blended with the remaining croscarmellose sodium, lubricated with the magnesium stearate, and compressed into tablets. The compression parameters were adjusted to achieve acceptable hardness, friability, and disintegration time.

TABLE 2
Composition of Formulations 2 and 3

| | Formulation 2 | Formulation 3 |
|---|---|---|
| Component | Amount (mg/tablet) | Amount (mg/tablet) |
| Compound A | 6.0 | 6.0 |
| Microcrystalline cellulose | 82.0 | 82.0 |
| Mannitol | 82.0 | 82.0 |
| Croscarmellose sodium | 18.0 | 18.0 |
| Sucrose palmitate | 10.0 | — |
| Poloxamer | — | 10.0 |
| Magnesium stearate | 2.0 | 2.0 |
| Total | 200.0 | 200.0 |

Formulations were subjected to the temperature and relative humidity conditions for a period of two weeks. In the table, “% Claim” refers to the percentage of Compound A remaining, as compared to the theoretical target. Based on these accelerated stability results, sucrose palmitate-containing formulations offered the better overall stability than poloxamer-containing formulations under the conditions studied (Table 3).

TABLE 3
Compound A Chemical Stability of Formulation
2 and Formulation 3 Tablets

| | | | Total |
|---|---|---|---|
| | | | Degradates |
| Formulation | Conditions | % Claim | (%) |
| Formulation 2 | −20° C. (closed) | 103.3 | 0.07 |
| | 30° C./65% RH (closed) | 105.3 | 0.22 |
| | 30° C./65% RH (open) | 102.8 | 0.81 |
| | 40° C./75% RH (closed) | 104.8 | 0.38 |
| | 40° C./75% RH (open) | 98.4 | 4.44 |
| | 40° C./50% RH (closed vial) | 103.1 | 0.90 |
| | 50° C./40% RH (closed vial) | 92.5 | 6.28 |
| Formulation 3 | −20° C. (closed) | 101.7 | 0.03 |
| | 30° C./65% RH (closed) | 102.4 | 0.07 |
| | 30° C./65% RH (open) | 100.9 | 0.53 |
| | 40° C./75% RH (closed) | 101.4 | 0.11 |
| | 40° C./75% RH (open) | 101.3 | 1.27 |
| | 40° C./50% RH (closed vial) | 101.4 | 0.52 |
| | 50° C./40% RH (closed vial) | 101.7 | 0.40 |

### Example 3: Roller-Compacted Formulations of Compound A

Formulations 4 and 5 are conventional roller compacted tablet formulations in which Compound A is prepared by a spray drying process, by spray drying from acetone. The spray-drying solution containing 10% solids was prepared. The spray-drying solution was atomized into a spray of droplets using a 0.6 mm bi-fluid nozzle. The spray dryer experiments were designed to achieve a specific target nitrogen gas outlet temperature (50° C.-60° C.). The solution feed rate (7 mL/min) was controlled by an external peristaltic pump. The atomizing nitrogen rate was 5 L/min. The spray-dried Compound A was dried at 40° C. under a vacuum.

The resulting spray-dried Compound A was blended with microcrystalline cellulose, mannitol, sucrose palmitate, or poloxamer, and two-thirds of the croscarmellose sodium and lubricated with half of the magnesium stearate. The resulting blends are compacted into slugs. The slugs were milled through a screen with an opening size of approximately 1 mm. The resulting granules are blended with the remaining croscarmellose sodium, lubricated with the magnesium stearate, and compressed into tablets. The compression parameters were adjusted to achieve acceptable hardness, friability, and disintegration time.

TABLE 4
Composition of Formulation 4 and Formulation 5

| | Formulation 4 | Formulation 5 |
|---|---|---|
| Component | Amount (mg/tablet) | Amount (mg/tablet) |
| Compound A | 60.0 | 60.0 |
| Microcrystalline cellulose | 55.0 | 55.0 |
| Mannitol | 55.0 | 55.0 |
| Croscarmellose sodium | 18.0 | 18.0 |
| Sucrose palmitate | 10.0 | — |
| Poloxamer | — | 10.0 |
| Magnesium stearate | 2.0 | 2.0 |
| Total | 200.0 | 200.0 |

The oral absorption obtained from Formulation 4 was determined in a preclinical pharmacokinetic study conducted in beagle dogs. In this study, 6 male beagle dogs were each dosed once. The results are shown in Tables 5 and 6.

TABLE 5
Summary of PK Results (Mean ± SE) for 60 mg Doses
of Compound A Administered as Formulation 4 or as
Control (Conventional Dry-Filled Capsule) Formulation
to Pentagastrin-Pre-Treated Male Beagle Dogs

| | Formulation 4 | Control |
|---|---|---|
| AUC_{0-∞} (nM · h) | 10,340 ± 1565 | 11,556 ± 2210 |
| Cₘₐₓ (nM) | 1,824 ± 199 | 1,705 ± 140 |

TABLE 6
Summary of PK Results (Mean ± SE) for 60 mg Doses
of Compound A Administered as Formulation 4 in Famotidine
Pre-Treated Male Beagle Dogs Dose, Compared to Control
(Formulation 4 in Pentagastrin Pre-Treated Male Beagle Dogs)

| | Formulation 4 | Control |
|---|---|---|
| AUC_{0-∞} (nM · h) | 1,770 ± 216 | 10,340 ± 1565 |
| Cₘₐₓ (nM) | 371 ± 33 | 1,824 ± 199 |

### Example 4: Roller-compacted Formulation of Compound A

Solid Dispersion Composition 1 is used in a tablet composition, Formulation 6, containing a spray-dried composition comprising Compound A, as shown in Table 7. The spray-dried composition was prepared from a solution comprising Compound A and HPMCAS by spray drying from acetone. The spray-drying solution containing 10% solids was prepared. The spray-drying solution was atomized into a spray of droplets using a 0.6 mm bi-fluid nozzle. The spray dryer experiments were designed to achieve a specific target nitrogen gas outlet temperature (50° C.-60° C.). The solution feed rate (7 ml/min) was controlled by an external peristaltic pump. The atomizing nitrogen rate was 5 L/min. The spray-dried Compound A composition was dried at 40° C. under a vacuum.

The resulting spray-dried composition was blended with the microcrystalline cellulose, mannitol, sucrose palmitate, and two-thirds of the croscarmellose sodium and lubricated with half of the magnesium stearate. The resulting blends are compacted into slugs. The slugs were milled through a screen with an opening size of approximately 1 mm. The resulting granules were blended with the remaining croscarmellose sodium, lubricated with the magnesium stearate, and finally compressed into tablets. The compression parameters were adjusted to achieve acceptable hardness, friability, and disintegration time.

TABLE 7
Composition of Formulation 6

| Component | Amount (mg/tablet) |
|---|---|
| Solid Dispersion Composition 1 | |
| Compound A | 60 |
| HPMCAS | 15 |
| Remaining Components | |
| Microcrystalline cellulose | 47.5 |
| Mannitol | 47.5 |
| Croscarmellose sodium | 18 |
| Sucrose palmitate | 10 |
| Magnesium stearate | 2 |
| Total | 200 |

### Example 5: Roller-Compacted Formulation of Compound A

Solid Dispersion Composition 2 is used in a tablet composition, Formulation 7, containing a spray-dried composition comprising Compound A as shown in Table 8. The spray-dried composition was prepared from a solution comprising Compound A and sucrose palmitate by spray drying from acetone. The spray-drying solution was prepared such that it contained 10% solids in solution. The spray-drying solution was atomized into a spray of droplets using a 0.6 mm bi-fluid nozzle. The spray dryer experiments were designed to achieve a specific target nitrogen gas outlet temperature (50° C.-60° C.). The solution feed rate (7 mL/min) was controlled by an external peristaltic pump. The atomizing nitrogen rate was 5 L/min. The spray-dried Compound A composition was dried at 40° C. under a vacuum.

The resulting spray-dried composition was blended with the microcrystalline cellulose, mannitol, and two-thirds of the croscarmellose sodium and lubricated with half of the magnesium stearate. The resulting blends are compacted into slugs. The slugs were milled through a screen with an opening size of approximately 1 mm. The resulting granules were blended with the remaining croscarmellose sodium, lubricated with the magnesium stearate, and finally compressed into tablets. The compression parameters were adjusted to achieve acceptable hardness, friability, and disintegration time.

TABLE 8
Composition of Formulation 7

| Component | Amount (mg/tablet) |
|---|---|
| Solid Dispersion Composition 2 | |
| Compound A | 60 |
| Sucrose palmitate | 6.7 |
| Remaining Components | |
| Microcrystalline cellulose | 56.7 |
| Mannitol | 56.6 |
| Croscarmellose sodium | 18 |
| Magnesium stearate | 2 |
| Total | 200 |

### Example 6: Roller-Compacted Formulation of Compound A

Solid Dispersion Composition 3 is used in a tablet composition, Formulation 8, containing a spray-dried composition comprising Compound A as shown in Table 9. The spray-dried composition was prepared from a solution comprising Compound A and HPMCAS by spray drying from acetone. The spray-drying solution was prepared such that it contained 10% solids in solution. The spray-drying solution was atomized into a spray of droplets using a 0.6 mm bi-fluid nozzle. The spray dryer experiments were designed to achieve a specific target nitrogen gas outlet temperature (50° C.-60° C.). The solution feed rate (7 mL/min) was controlled by an external peristaltic pump. The atomizing nitrogen rate was 5 L/min. The spray-dried Compound A composition was dried at 40° C. under a vacuum.

The resulting spray-dried composition was blended with the microcrystalline cellulose, mannitol, sucrose palmitate, and two-thirds of the croscarmellose sodium and lubricated with half of the magnesium stearate. The resulting blends are compacted into slugs. The slugs were milled through a screen with an opening size of approximately 1 mm. The resulting granules were blended with the remaining croscarmellose sodium, lubricated with the magnesium stearate, and finally compressed into tablets. The compression parameters were adjusted to achieve acceptable hardness, friability, and disintegration time.

TABLE 9
Composition of Formulations 8

| Component | Amount (mg/tablet) |
|---|---|
| Solid Dispersion Composition 3 | |
| Compound A | 60 |
| HPMCAS | 40 |
| Remaining Components | |
| Microcrystalline cellulose | 35.0 |
| Mannitol | 35.0 |
| Croscarmellose sodium | 18 |
| Sucrose palmitate | 10 |
| Magnesium stearate | 2 |
| Total | 200 |

### Example 7: Wet-Granulated Formulation of Compound A

Formulation 9 is a conventional wet-granulated tablet formulation in which Compound A is formulated as a pure amorphous Compound A (Table 10). Compound A was blended with microcrystalline cellulose, mannitol, hydroxypropyl cellulose, sucrose palmitate, and two-thirds of the croscarmellose sodium, added to the bowl of a high-shear granulator, and granulated with a TPGS solution in water. The resulting granules were dried, milled through a screen with an opening size of approximately 0.8 mm, blended with the remaining croscarmellose sodium, lubricated with the magnesium stearate, and compressed into tablets. The compression parameters were adjusted to achieve acceptable hardness, friability, and disintegration time.

For the following formulations, amorphous Compound A as received were used.

TABLE 10
Composition of Formulation 9

| Component | Amount (mg/tablet) |
|---|---|
| Compound A | 60.0 |
| Microcrystalline cellulose | 47.5 |
| Mannitol | 47.5 |
| Hydroxypropylcellulose | 6.0 |
| Croscarmellose sodium | 18.0 |
| TPGS | 16.0 |
| Sucrose palmitate | 4.0 |
| Magnesium stearate | 1.0 |
| Total | 200.0 |

The oral absorption obtained from Formulation 9 was determined in a preclinical pharmacokinetic study conducted in beagle dogs. In this study. 6 male beagle dogs were each dosed once. The results are shown in Table 11.

TABLE 11
Summary of PK Results (Mean ± SE) for 60 mg Doses
of Compound A Administered as Formulation 9 or as
Control (Conventional Dry-Filled Capsule) Formulation
to Pentagastrin-Pre-Treated Male Beagle Dogs

| | Formulation 9 | Control |
|---|---|---|
| AUC_{0-∞} (nM · h) | 9,205 ± 409 | 11,556 ± 2210 |
| Cₘₐₓ (nM) | 1,844 ± 68 | 1,705 ± 140 |

TABLE 12
Summary of PK Results (Mean ± SE) for 60 mg Doses
of Compound A Administered as Formulation 9 in Famotidine
Pre-Treated Male Beagle Dogs Dose, Compared to Control
(Formulation 9 in Pentagastrin Pre-Treated Male Beagle Dogs)

| | Formulation 9 | Control |
|---|---|---|
| AUC_{0-∞} (nM · h) | 3587 ± 589 | 9,205 ± 409 |
| Cₘₐₓ (nM) | 595 ± 46 | 1,844 ± 68 |

### Example 8: Wet-Granulated Formulation of Compound A

Formulation 10 is a conventional wet-granulated tablet formulation in which Compound A is formulated as a pure amorphous Compound A (Table 13). Compound A was blended with microcrystalline cellulose, lactose, hydroxypropyl cellulose, and two-thirds of the croscarmellose sodium, added to the bowl of a high-shear granulator, and granulated with a TPGS solution in water. The resulting granules were dried, milled through a screen with an opening size of approximately 0.8 mm, blended with the remaining croscarmellose sodium, lubricated with the magnesium stearate, and compressed into tablets. The compression parameters were adjusted to achieve acceptable hardness, friability, and disintegration time.

TABLE 13
Composition of Formulation 10

| Component | Amount (mg/tablet) |
|---|---|
| Compound A | 60.0 |
| Microcrystalline cellulose | 52.5 |
| Lactose | 52.5 |
| Hydroxypropylcellulose | 6.0 |
| Croscarmellose sodium | 18.0 |
| TPGS | 10.0 |
| Magnesium stearate | 1.0 |
| Total | 200.0 |

### Example 9: Wet-Granulated Formulation of Compound A

Formulation 11 is a conventional wet-granulated tablet formulation in which Compound A is formulated as a pure amorphous Compound A (Table 14). Compound A was blended with microcrystalline cellulose, lactose, hydroxypropyl cellulose, the sucrose palmitate, and two-thirds of the croscarmellose sodium, added to the bowl of a high-shear granulator, and granulated in water. The resulting granules were dried, milled through a screen with an opening size of approximately 0.8 mm, blended with the remaining croscarmellose sodium, lubricated with the magnesium stearate, and compressed into tablets. The compression parameters were adjusted to achieve acceptable hardness, friability, and disintegration time.

TABLE 14
Composition of Formulation 11

| Component | Amount (mg/tablet) |
|---|---|
| Compound A | 60.0 |
| Microcrystalline cellulose | 52.5 |
| Lactose | 52.5 |
| Hydroxypropylcellulose | 6.0 |
| Croscarmellose sodium | 18.0 |
| Sucrose palmitate | 10.0 |
| Magnesium stearate | 1.0 |
| Total | 200.0 |

### Example 10: Wet-Granulated Formulation of Compound A

Formulation 12 is a conventional wet-granulated tablet formulation in which Compound A is formulated as a pure amorphous Compound A (Table 15). Compound A was blended with microcrystalline cellulose, lactose, hydroxypropyl cellulose, and two-thirds of the croscarmellose sodium, added to the bowl of a high-shear granulator, and granulated with a polyoxyethylene (20) sorbitan monooleate/sorbitan monooleate solution in water. The resulting granules were dried, milled through a screen with an opening size of approximately 0.8 mm, blended with the remaining croscarmellose sodium, lubricated with the magnesium stearate, and compressed into tablet. The compression parameters were adjusted to achieve acceptable hardness, friability, and disintegration time.

TABLE 15
Composition of Formulation 12

| Component | Amount (mg/tablet) |
|---|---|
| Compound A | 60.0 |
| Microcrystalline cellulose | 52.5 |
| Lactose | 52.5 |
| Hydroxypropylcellulose | 6.0 |
| Croscarmellose sodium | 18.0 |
| Polyoxyethylene (20) sorbitan monooleate | 4.0 |
| Sorbitan monooleate | 6.0 |
| Magnesium stearate | 1.0 |
| Total | 200.0 |

### Example 11: Wet-Granulated Formulation of Compound A

Formulation 13 is a conventional wet-granulated tablet formulation in which Compound A is prepared by a spray drying process. The spray-dried intermediate was prepared from a solution comprising Compound A by spray drying from acetone. The spray-drying solution was prepared such that it contained 10% solids in solution. The spray-drying solution was atomized into a spray of droplets using a 0.6 mm bi-fluid nozzle. The spray dryer experiments were designed to achieve a specific target nitrogen gas outlet temperature (50° C.-60° C.). The solution feed rate (7 mL/min) was controlled by an external peristaltic pump. The atomizing nitrogen rate was 5 L/min. The spray-dried Compound A was dried at 40° C. under a vacuum.

The resulting spray-dried Compound A was blended with microcrystalline cellulose, lactose, hydroxypropyl cellulose, and two-thirds of the croscarmellose sodium, added to the bowl of a high-shear granulator, and granulated with a polyoxyethylene (20) sorbitan monooleate/sorbitan monooleate solution in water. The resulting granules were dried, milled through a screen with an opening size of approximately 0.8 mm, blended with the remaining croscarmellose sodium, lubricated with the magnesium stearate, and compressed into tablets. The compression parameters were adjusted to achieve acceptable hardness, friability, and disintegration time.

TABLE 16
Composition of Formulation 13

| Component | Amount (mg/tablet) |
|---|---|
| Compound A | 60.0 |
| Microcrystalline cellulose | 52.5 |
| Lactose | 52.5 |
| Hydroxypropylcellulose | 6.0 |
| Croscarmellose sodium | 18.0 |
| Polyoxyethylene (20) sorbitan monooleate | 4.0 |
| sorbitan monooleate | 6.0 |
| Magnesium stearate | 1.0 |
| Total | 200.0 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A tablet comprising a pharmaceutical formulation comprising:
(a) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, Compound A: 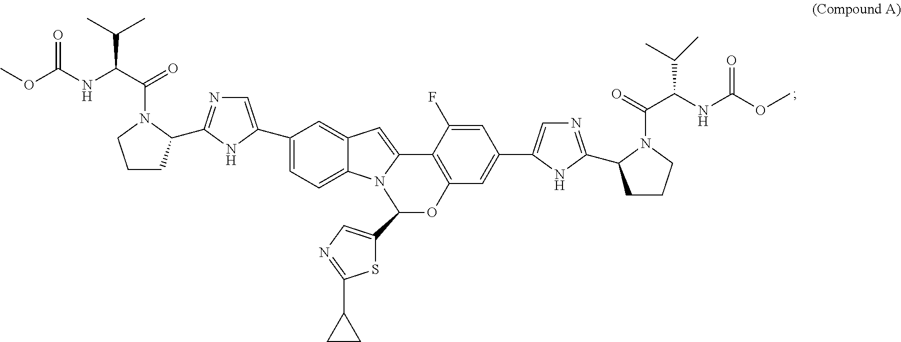
and one or more of:
(b) a pharmaceutically acceptable diluent;
(c) a pharmaceutically acceptable disintegrant;
(d) a pharmaceutically acceptable lubricant;
(e) a pharmaceutically acceptable glidant;
(f) a pharmaceutically acceptable surfactant; and
(g) a pharmaceutically acceptable polymer,
wherein Compound A is substantially amorphous, said pharmaceutical formulation is prepared by roller compaction, and said tablet does not comprise an ionic salt.

2. The tablet of claim 1, wherein Compound A is provided as
(i) a spray-dried composition comprising substantially amorphous Compound A and a pharmaceutically acceptable polymer,
(ii) a spray-dried composition comprising substantially amorphous Compound A and a pharmaceutically acceptable surfactant, or
(iii) a spray-dried composition comprising substantially amorphous Compound A, a pharmaceutically acceptable polymer, and a pharmaceutically acceptable surfactant.

3. The tablet according to claim 1, wherein Compound A is present in a total concentration of from about 3% w/w to about 45% w/w.

4. The tablet of claim 1, wherein the pharmaceutically acceptable diluent is selected from the group consisting of mannitol, microcrystalline cellulose, and lactose, and combinations thereof, and wherein said pharmaceutically acceptable diluent is present in a total concentration of from about 3% w/w to about 60% w/w.

5. The tablet of claim 1, wherein the pharmaceutically acceptable disintegrant is selected from the group consisting of croscarmellose sodium, crospovidone, other celluloses, and mixtures thereof, and wherein said pharmaceutically acceptable disintegrant is present in a total concentration of from about 4% w/w to about 20% w/w.

6. The tablet of claim 1, wherein the pharmaceutically acceptable lubricant is selected from the group consisting of calcium stearate, magnesium stearate, stearic acid, zinc stearate, and mixtures thereof, and wherein said pharmaceutically acceptable lubricant is present in a total concentration of from about 0.5% w/w to about 4% w/w.

7. The tablet of claim 1, wherein the pharmaceutically acceptable glidant is selected from the group consisting of starch, talc, magnesium stearate, and silicon dioxide, and combinations thereof, and wherein said pharmaceutically acceptable glidant is present in a total concentration of from about 0% w/w to about 2% w/w.

8. The tablet of claim 1, wherein the pharmaceutically acceptable surfactant is selected from the group consisting of sorbitan fatty acid mono esters D-alpha-tocopheryl polyethylene glycol 1000 succinate (TPGS), poloxamer, sucrose palmitate, sorbitan oleate, polyoxyethyl (20) sorbitan monooleate, block copolymers of ethylene oxide and propylene oxide, and combinations thereof, and wherein said pharmaceutically acceptable surfactant is present in a total concentration of from about 0% w/w to about 2% w/w.

9. A tablet comprising a pharmaceutical formulation comprising:
(a) dimethyl ((2S,2′S)-((2S,2′S)-2,2′-(5,5′-((S)-6-(2-cyclopropylthiazol-5-yl)-1-fluoro-6H-benzo[5,6][1,3]oxazino[3,4-a]indole-3,10-diyl)bis(1H-imidazole-5,2-diyl))bis(pyrrolidine-2,1-diyl))bis(3-methyl-1-oxobutane-2,1-diyl))dicarbamate, Compound A: 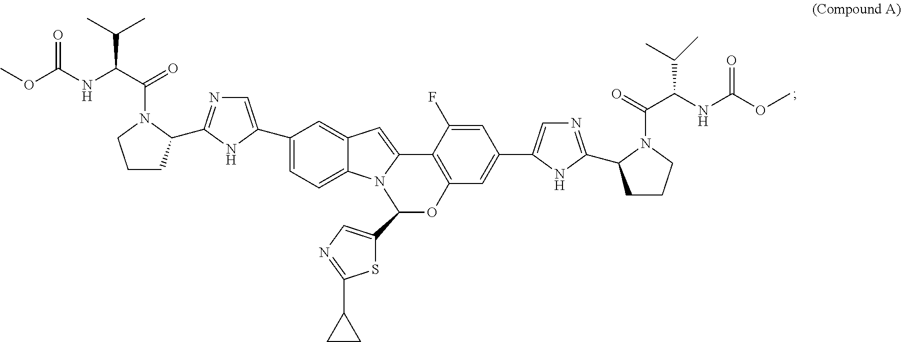
and one or more of:
(b) a pharmaceutically acceptable diluent;
(c) a pharmaceutically acceptable disintegrant;
(d) a pharmaceutically acceptable lubricant;
(e) a pharmaceutically acceptable glidant;
(f) a pharmaceutically acceptable surfactant; and
(g) a pharmaceutically acceptable polymer,
wherein Compound A is substantially amorphous, said pharmaceutical formulation is prepared by wet-granulation, and said tablet does not comprise an ionic salt.

10. The tablet of claim 9, wherein Compound A is provided directly from synthesis or as spray-dried compound.

11. The tablet of claim 9, wherein Compound A is present in a total concentration of from about 3% w/w to about 45% w/w.

12. The tablet of claim 9, wherein the pharmaceutically acceptable diluent is selected from the group consisting of mannitol, microcrystalline cellulose, and lactose, and combinations thereof, and wherein said pharmaceutically acceptable diluent is present in a total concentration of from about 3% w/w to about 60% w/w.

13. The tablet of claim 9, wherein the pharmaceutically acceptable disintegrant is selected from the group consisting of croscarmellose sodium, crospovidone, other celluloses, and mixtures thereof, and wherein said pharmaceutically acceptable disintegrant is present in a total concentration of from about 4% w/w to about 20% w/w.

14. The tablet of claim 9, wherein the pharmaceutically acceptable lubricant is selected from the group consisting of calcium stearate, magnesium stearate, stearic acid, zinc stearate, and mixtures thereof, and wherein said pharmaceutically acceptable lubricant is present in a total concentration of from about 0.5% w/w to about 4% w/w.

15. The tablet of claim 9, wherein the pharmaceutically acceptable glidant is selected from the group consisting of starch, talc, magnesium stearate, and silicon dioxide, and combinations thereof, and wherein said pharmaceutically acceptable glidant is present in a total concentration of from about 0% w/w to about 2% w/w.

16. The tablet of claim 9, wherein the pharmaceutically acceptable surfactant is selected from the group consisting of sorbitan fatty acid mono esters D-alpha-tocopheryl polyethylene glycol 1000 succinate (TPGS), poloxamer, sucrose palmitate, sorbitan oleate, polyoxyethyl (20) sorbitan monooleate, block copolymers of ethylene oxide and propylene oxide, and combinations thereof, and wherein said pharmaceutically acceptable surfactant is present in a total concentration of from about 0% w/w to about 2% w/w.

17. The tablet of claim 9, further comprising a solubilizer, wherein the solubilizer is present in a concentration of from 2% w/w to about 15% w/w.

18. The tablet of claim 9, further comprising a wetting agent, wherein the wetting agent is present in a concentration of from about 1% w/w to about 10% w/w.

19. The tablet of claim 1, wherein the pharmaceutically acceptable polymer is selected from the group consisting of hydroxypropyl cellulose, HPMC, HPMCAS, and combinations thereof, and wherein said pharmaceutically acceptable polymer is present in a total concentration of from about 40% w/w to about 99.9% w/w.

20. The tablet of claim 9, wherein the pharmaceutically acceptable polymer is selected from the group consisting of hydroxypropyl cellulose, HPMC, HPMCAS, and combinations thereof, and wherein said pharmaceutically acceptable polymer is present in a total concentration of from about 40% w/w to about 99.9% w/w.
